(19) United States
(12) Patent Application Publication
(10) Pub. No.: US 2025/0339590 A1
(43) Pub. Date: Nov. 6, 2025

(54) DECELLULARIZED PORCINE SEROSAL BIOMATERIAL AND APPLICATIONS AND USES THEREOF

(71) Applicant: Arizona Board of Regents on behalf of Arizona State University, Scottsdale, AZ (US)

(72) Inventors: Kaushal Rege, Gilbert, AZ (US); Vanshika Singh, Scottsdale, AZ (US); Muhammad Raisul Abedin, Indianapolis, IN (US); Jordan Yaron, Scottdale, AZ (US)

(21) Appl. No.: 19/196,318

(22) Filed: May 1, 2025

Related U.S. Application Data
(60) Provisional application No. 63/641,379, filed on May 1, 2024.

(51) Int. Cl. A61L 31/14; A61L 31/00; C12N 9/76

(52) U.S. Cl. CPC A61L 31/145; A61L 31/005; C12N 9/6427; A61L 2430/40; C12Y 304/21004

(57) ABSTRACT

The present disclosure relates to an extracellular matrix-based biomaterial that allows for the prevention of post-operative adhesions. The disclosed extracellular matrix-based biomaterial is produced from decellularized porcine small intestinal serosa. Methods of producing the extracellular matrix-based biomaterial from decellularized porcine small intestinal serosa are also disclosed.

### RELATED APPLICATIONS

This application claims the benefit of U.S. provisional patent application 63/641,379, filed May 1, 2024 to Rege et al., titled “DECELLULARIZED PORCINE SEROSAL BIOMATERIAL AND APPLICATIONS AND USES THEREOF,” the entirety of the disclosure of which is hereby incorporated by reference.

### STATEMENT REGARDING FEDERALLY SPONSORED RESEARCH OR DEVELOPMENT

This invention was made with government support under EB020690 and AR074627 awarded by the National Institutes of Health. The government has certain rights in the invention.

### TECHNICAL FIELD

The present disclosure relates to a biomaterial derived from porcine small intestine serosa.

### BACKGROUND

Post-operative peritoneal adhesions (PPA), also known as intra-abdominal adhesions, pose a major challenge in the field of gastrointestinal surgery. Post-operative adhesions develop as a result of the body's response to a tissue trauma following surgery, but they can also develop as a consequence of peritoneal tissue irritation caused by infection. Regardless of the procedure technique or its location in the body, intra-abdominal adhesions occur in majority of the patients undergoing surgical procedures. In many cases these post-operative peritoneal adhesions remain asymptomatic, but they can cause numerous secondary problems like chronic abdominal pain, recurrent intestinal obstructions, infertility, chronic morbidity with no effective therapy, and mortality. In the US healthcare system alone, over $2.5 billion is spent in the treatment of post-surgical adhesions, and complications related to post-surgical adhesions result in approximately one million days of additional inpatient care annually. Moreover, expenditures associated with diagnostics, imaging, laboratory tests, long-term morbidity etc. add to the costs, exponentially increasing the already staggering estimate.

Decellularization is the process of removing cells and their parts from the extracellular matrix (ECM), specifically DNA and RNA, to produce a natural matrix with preserved integrity. Decellularized tissues have been used in an array of applications such as tissue engineering, cell transplantation, in vitro & in vivo modeling, therapeutic delivery vehicles as well as in clinical applications like wound healing products, surgical meshes/grafts etc.

### SUMMARY

In some aspects, the disclosure relates to a barrier material comprising at least two layers of decellularized extracellular matrix (dECM) from a porcine small intestine serosal tissue, wherein the barrier material is a sheet including at least two layers of dECM from a porcine small intestine serosa, and wherein the layers of dECM are arranged on top of each other.

In some aspects, the barrier material is a sheet including 4-8 layers of dECM from a porcine small intestine serosa.

In some aspects of the barrier material, each layer of dECM from porcine small intestine serosa is conjugated with at least one of: a carboxylic acid, an amine, a hydrophilic polymer, and/or a combination thereof.

In some embodiments of the barrier material, the sheet is lyophilized.

In some aspects, the disclosure relates to a method of decellularizing a serosal layer from a porcine small intestine, the method comprising: isolating the serosal layer from the porcine small intestine; incubating the isolated serosal layer in a solution including chloroform and methanol for at least 8 hours to produce a degreased serosal layer; incubating the degreased serosal layer with an enzyme solution for at least 8 hours to produce a partially decellularized serosal layer; incubating the partially decellularized serosal layer with a detergent solution under agitation for at least two hours to produce decellularized serosal layer; and incubating the decellularized serosal layer with peracetic acid solution followed by ethanol solution to produce a dECM from porcine small intestine serosa.

In some aspects of the method of decellularizing a serosal layer from a porcine small intestine, the ratio of chloroform to methanol in the solution used to produce a degreased serosal layer is 1:1 by volume.

In some aspects of the method of decellularizing a serosal layer from a porcine small intestine, the enzyme solution includes 0.05-0.5% trypsin.

In some aspects of the method of decellularizing a serosal layer from a porcine small intestine, the detergent solution includes 0.1-1% sodium dodecyl sulfate (SDS).

In some implementations of the method of decellularizing a serosal layer from a porcine small intestine, the partially decellularized serosal layer is incubated in the detergent solution on an orbital shaker.

In some aspects, the disclosure relates to a method of producing a barrier material. The method comprises the above described method of decellularizing a serosal layer from a porcine small intestine and the steps of layering the dECM from the porcine small intestine serosa to produce a multi-layered dECM product; and lyophilizing the multi-layered dECM product to produce the barrier material.

In some aspects, the method of producing a barrier material further comprises: producing a chemically modified dECM by conjugating the dECM from the porcine small intestine serosa with at least one of a carboxylic acid, an amine, a hydrophilic polymer, and/or a combination thereof, wherein the chemically modified dECM is layered to produce the multi-layered dECM product.

In some aspects, the method of producing a barrier material further comprises subjecting the dECM from the porcine small intestine serosa to an ethylene oxide treatment.

In some implementations of the method of producing a barrier material, eight layers of the dECM from the porcine small intestine serosa are layered to produce the multi-layered dECM product.

In some aspects, the disclosure relates to a method of producing a hydrogel. The method comprises the above described method of producing a barrier material and the step of pulverizing the dECM from the porcine small intestine serosa to produce a powdered decellularized serosal layer; digesting the powdered decellularized serosal layer with an enzyme to produce a hydrogel precursor solution, wherein the enzyme is selected from pepsin, papain, amylase, or collagenase; lyophilizing the hydrogel precursor solution to produce a sponge-like matrix; and pulverizing the sponge-like matrix to produce a hydrogel precursor powder.

In some aspects, the method of producing a hydrogel further comprises subjecting the dECM from the porcine small intestine serosa to an ethylene oxide treatment, thereby sterilizing the dECM from the porcine small intestine serosa.

In some aspects, the method of producing a hydrogel further comprises adding a buffered saline solution to the hydrogel precursor powder to produce the hydrogel.

In some aspects, the disclosure relates to a hydrogel composition including: a decellularized extracellular matrix derived from a porcine small intestine serosal tissue, wherein the decellularized extracellular matrix has been pulverized; and a liquid, wherein the hydrogel composition is gel-like at a temperature of at least 35° C.

In some embodiments of the hydrogel composition, the buffered saline solution is a phosphate-buffered saline and the pH of the hydrogel composition is neutral.

In some aspects, the disclosure relates to a kit for preventing post-operative intestinal adhesions comprising dECM from a porcine small intestine serosal tissue.

The foregoing and other aspects, features, and advantages will be apparent from the DESCRIPTION and DRAWINGS, and from the CLAIMS if any are included.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements.

The features and advantages of the present disclosure, and the manner of attaining them, will become more apparent and the present disclosure will be better understood by reference to the description of the present disclosure taken in conjunction with the accompanying drawings, wherein:

FIG. 1 depicts, in accordance with certain embodiments, an exemplary process for decellularizing porcine intestinal serosa for the preparation of a barrier material.

FIGS. 2A and 2B depict exemplary embodiments comparing swelling ratio (FIG. 2A) and anti-adhesive behavior (FIG. 2B) of Serografts (produced from porcine intestinal serosa according to the method disclosed herein) with that of Cook Biotech™ SIS, Seprafilm®, and a chistosan-based biomaterial film developed in the laboratory (Chitosan LASE).

FIGS. 3A and 3B depict exemplary embodiments comparing ultimate tensile strength (FIG. 3A) and Young's modulus (FIG. 3B) of Serografts (produced from porcine intestinal serosa according to the method disclosed herein) with that of Cook Biotech™ SIS, Seprafilm®, and Chitosan LASE.

FIGS. 4A-4C depict exemplary embodiments comparing dynamic mechanical properties of Serografts (produced from porcine intestinal serosa according to the method disclosed herein) with that of Cook Biotech™ SIS, Seprafilm®, and Chitosan LASE. Storage modulus is depicted in FIG. 4A. Loss modulus is depicted in FIG. 4B. Ratio of loss to storage modulus (tan δ) is depicted in FIG. 4C.

FIGS. 5A and 5B depict scanning electron microscopy images of Serografts prepared from intestinal serosal tissue of four different animals at 4000× (FIG. 5A) and 15000× (FIG. 5B) magnification.

FIGS. 6A-6F depict exemplary histological analysis (FIGS. 6A-6C) images of Serografts and fluorescent microscopy images of Serografts (FIGS. 6D-6F) identifying collagen IV, elastin, and laminin presence and location using immunofluorescence.

FIG. 7 depicts the experimental plan for studying the efficacy of the Serograft disclosed herein on preventing intra-abdominal adhesions.

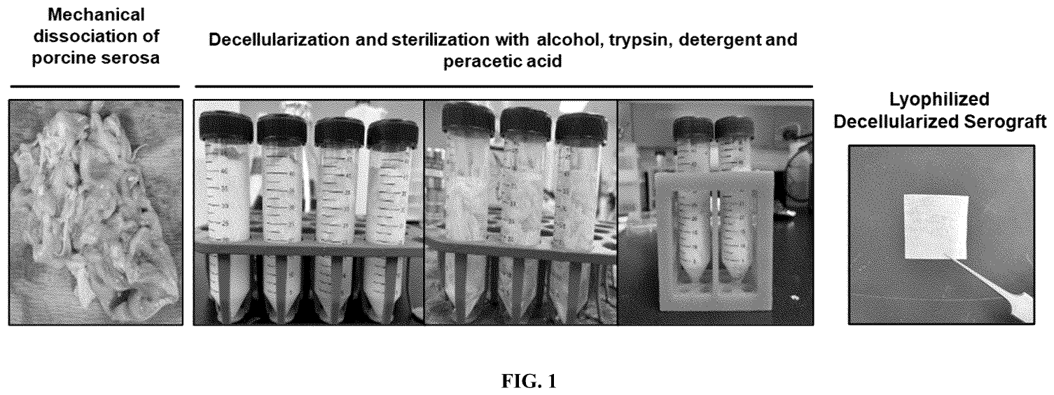

Those of ordinary skill in the art will understand that the compositions, methods, kits, and systems specifically described herein and illustrated in the accompanying drawings are non-limiting exemplary embodiments and that the scope of the various embodiments of the present disclosure is defined solely by the claims. The features illustrated or described in connection with one exemplary embodiment may be combined with the features of other embodiments. Such modifications and variations are intended to be included within the scope of the present disclosure.

### DETAILED DESCRIPTION

Detailed aspects and applications of the disclosure are described below in the drawings and detailed description of the disclosure. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the disclosure. It will be understood, however, by those skilled in the relevant arts, that the present disclosure may be practiced without these specific details. It should be noted that there are many different and alternative configurations, devices, and technologies to which the disclosed disclosures may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps.

As used herein, the term “about” to when used in the context of numeric values denotes an interval of accuracy, familiar, and acceptable to a person skilled in the art. Said interval can be ±2% of the given value, ±5%, or ±10% of the numeric values, where applicable. In some aspects, said interval describes standard deviations of ±2%. In some aspects, said interval describes standard deviations of ±5%. In some aspects, said interval describes standard deviations of ±10%.

As used herein, the term “abdominal surgery” refers to a procedure performed on the abdominal cavity. In some aspects, the procedure treats or diagnoses a condition affecting the digestive tract, liver, gallbladder, pancreas, appendix, spleen, and/or surrounding tissues. The term “abdominal surgery” includes a traditional open surgery as well as minimally invasive surgery.

As used herein, the term “subject” refers to any organism to which a provided barrier material is or may be administered, e.g., for experimental, diagnostic, prophylactic, cosmetic, band/or therapeutic purposes. Typical subjects include animals (e.g., mammals such as mice, rats, rabbits, non-human primates, and/or humans). In some embodiments, a subject is a human. In some embodiments, a subject is suffering from or susceptible to one or more disorders or conditions involving the digestive tract, liver, gallbladder, pancreas, appendix, spleen, and/or surrounding tissues. In some embodiments, a subject displays one or more symptoms of a disorder or condition involving the digestive tract, liver, gallbladder, pancreas, appendix, spleen, and/or surrounding tissues. In some embodiments, a subject has been diagnosed with one or more disorders or conditions involving the digestive tract, liver, gallbladder, pancreas, appendix, spleen, and/or surrounding tissues. In some embodiments, the disorder or condition is or includes peritonitis, a ruptured organ (e.g., appendix, stomach), diverticulitis, Crohn's disease, pelvic inflammatory disease, a cancer, peptic ulcer perforation, and/or peritoneal dialysis complications. In some embodiments, the subject is receiving or has received an abdominal surgery to diagnose and/or treat a disease, disorder, or condition.

This disclosure is directed to a decellularized extracellular matrix (dECM) derived from porcine a small intestine serosal tissue (also referred to herein as “Serograft”).

The outer covering of the organs and body cavities in the chest and abdomen, including the stomach, is lined by a layer called the serosal layer, which is also known as the serous membrane. The serosal layer is a smooth tissue membrane made of mesothelium and has anti-adhesive properties. It secretes serous fluid, allowing lubricated sliding movement between opposing surfaces of internal organs. As shown in the Examples, the novel extracellular matrix-based biomaterial derived from the porcine small intestine serosa preserves the serosal tissue's inherent anti-adhesive qualities. In some embodiments, a dECM from a porcine small intestine serosa suitable for use as a tissue graft material is disclosed. In some aspects, the dECM from the porcine small intestine serosa is also useful for preventing post-operative intestinal adhesions. In some such embodiments, preventing post-operative intestinal adhesions may encompass reducing the total instances of post-operative intestinal adhesions.

The key proteins present in the dECM from the porcine small intestine serosa are listed in Table 1. In some aspects, the dECM is modified chemically to further augment the anti-adhesive qualities of the material. Several strategies can be adopted for performing the chemical modifications, including but not limited to, conjugation of a linear polyethylene glycol (PEG) molecule, a branched PEG molecule, and/or a combination thereof to the biomaterial using bioconjugation chemistries (e.g., EDC-NHS, thiolation) and/or click chemistries (e.g., copper-catalyzed azide-alkyne cycloaddition (CuAAC)).

TABLE 1
Key constituents of the dECM from the porcine small intestine serosa.
Constituents were identified from mass spectrometry performed at
the mass spectrometry facility at Arizona State University.

| Accession # | Protein | Gene |
|---|---|---|
| A0A287B5W2 | Trypsinogen isoform X1 | LOC100302368 |
| A0A287A1S6 | Collagen alpha-1(I) chain preproprotein | COL1A1 |
| A0A8X9AEL6 | Collagen type I alpha 2 chain | COL1A2 |
| I3LDS3 | Keratin, type I cytoskeletal 10 | KRT10 |
| A0A287A5G1 | Actin, cytoplasmic 2 | ACTG1 |
| F1SGG3 | Keratin, type II cytoskeletal 1 | KRT1 |
| A0A287BAY9 | Albumin | ALB |
| A0A8W4FNX1 | Actin, gamma-enteric smooth muscle | ACTG2 |
| A0A287AEL2 | IF rod domain-containing protein | LOC110255312 |
| A0A287ASK1 | Myosin-11 | MYH11 |
| A0A481AWD4 | NAD-capped RNA hydrolase NUDT12 | NUDT12 |
| A0A287BHY5 | Keratin 2 | KRT2 |
| A0A5G2RBD3 | Keratin 18 | KRT18 |
| F1S553 | Phospholipid phosphatase related 4 | PLPPR4 |
| I3LQ84 | Collagen type VI alpha 2 chain | COL6A2 |
| P02067 | Hemoglobin subunit beta | HBB |
| A0A286ZRI9 | MAGE domain-containing protein | n.a. |
| F1SGI7 | Keratin 75 | KRT75 |
| A0A287AXX7 | PRELI domain containing protein 3B | PRELID3B |
| A0A5G2RJ26 | Cyclin dependent kinase 16 | CDK16 |
| A0A287AD33 | WD repeat domain 17 | WDR17 |
| A0A287AG48 | Keratin, type II cytoskeletal 7 | KRT7 |
| A0A287BQ02 | Polyadenylate-binding protein | PABPC4 |
| A0A5G2QB91 | Mitochondrial-processing peptidase subunit alpha | PMPCA |
| A0A286ZVV4 | Ubiquitin interaction motif containing 1 | UIMC1 |
| F1SSR2 | Dispatched RND transporter family member 2 | DISP2 |
| A0A8W4FPR2 | Alanine--tRNA ligase | AARS1 |
| A0A8W4FCB4 | Golgin A4 | GOLGA4 |
| A0A5G2R7K7 | Integrin beta | ITGB3 |
| F1S6Z1 | BMP/retinoic acid inducible neural specific 2 | BRINP2 |
| A0A5G2QFH4 | Transformer 2 beta homolog | TRA2B |
| F1RJU6 | Neurofilament medium polypeptide | NEFM |
| A0A287AM15 | Synaptotagmin like 2 | SYTL2 |
| A0A5G2RHS3 | Mitochondrial potassium channel ATP-binding subunit | ABCB8 |
| A0A287B5P8 | Membrane associated guanylate kinase, WW and PDZ | MAGI1 |
| | domain containing 1 | |
| A0A286ZLN9 | N-alpha-acetyltransferase 16, NatA auxiliary subunit | NAA16 |
| Q59IP2 | Collagen alpha-2(V) chain | COL5A2 |
| A0A4X1WCZ6 | AT-rich interaction domain 4A | ARID4A |
| F1RL25 | Thioredoxin domain containing 11 | TXNDC11 |
| A0A287A935 | Heat shock protein family A (Hsp70) member 12B | HSPA12B |
| F1SUF3 | hexokinase | HKDC1 |
| A0A5G2REY7 | Collagen type IV alpha 2 chain | COL4A2 |
| A0A5G2QFG2 | Zinc finger protein 451 | ZNF451 |
| A0A8W4FES9 | Tropomyosin 1 | TPM1 |
| A0A287AZP6 | ERCC excision repair 6 like 2 | ERCC6L2 |
| A0A286ZY68 | IF rod domain-containing protein | KRT6A |
| A0A8W4FG22 | Peptidase S1 domain-containing protein | n.a. |
| A0A5G2QPA0 | Centromere protein J | CENPJ |
| A0A480I6I0 | Collagen alpha-1(XIV) chain | COL14A1 |
| I3LEX8 | Cytochrome P450 3A | CYP3A22 |
| F1RX65 | Transcription initiation factor IIE subunit beta | GTF2E2 |
| P01846 | Ig lambda chain C region | n.a. |
| I3L865 | CHD1 helical C-terminal domain containing 1 | CHCT1 |
| A0A287BIW9 | Coiled-coil domain containing 18 | CCDC18 |
| F1SDE8 | Tektin | TEKT3 |
| F2Z5L5 | Histone H2A | H2AC20 |
| I3LDT6 | Ovochymase 2 | OVCH2 |

In one aspect, a sheet of barrier material comprising at least two layers of dECM from porcine small intestine serosa is disclosed. The sheet of barrier material is also referred to herein as “Serograft.” In some embodiments, each layer of dECM from porcine small intestine serosa is translucent and stretched to form an even layer. In some embodiments, the dECM from the porcine small intestine serosa is visually similar to it native state. In some embodiments, the sheet of barrier material comprises two layers, four layers, six layers, or eight layers of dECM from the porcine small intestine serosa. In some aspects, the layers of dECM from the porcine small intestine serosa are cross-linked through chemical modifications for stronger bonding between the layers. In exemplary embodiments, carboxylic acids and amines are conjugated to surfaces of the layers of dECM from the porcine small intestine serosa to enable crosslinking between successive layers. In some implementations, carboxylic acids and amines are conjugated to surfaces of the layers of dECM from porcine small intestine serosa through EDC/NHS chemistry. In some aspects, the Serograft is chemically modified by conjugation with a hydrophilic polymer. In some embodiments, the hydrophilic polymer is selected from poly (2-hydroxyethyl methacrylate) (PHEMA) and poly (sulfobetaine methacrylate) (PSBMA). In some embodiments, the hydrophilic polymer is PHEMA. In some embodiments, the hydrophilic polymer is PSBMA. In some embodiments, the conjugation of the hydrophilic polymer to the Serograft reduces cell adhesion. In some embodiments, the hydrophilic polymers mimic the hydration properties of a PEG molecule. In some embodiments, each layer of the dECM from the porcine small intestine serosa is conjugated with at least one of: carboxylic acids, amines, a hydrophilic polymer, and/or a combination thereof.

In particular embodiments, the sheet of barrier material is lyophilized. In some embodiments, lyophilization takes place after the layers of dECM from the porcine small intestine serosa are arranged on top of each other to produce a multi-layered dECM from porcine small intestine serosa product. In certain implementations, the sheet of barrier material is produced from freezing the multi-layered dECM from porcine small intestine serosa product at −80° C. for a minimum of about 6 hours prior to lyophilization. In some embodiments, the sheet of barrier material is produced from freezing the multi-layered dECM from porcine small intestine serosa product at −80° C. for a minimum of about 7 hours prior to lyophilization. In some embodiments, the sheet of barrier material is produced from freezing the multi-layered dECM from porcine small intestine serosa product at −80° C. for a minimum of about 8 hours prior to lyophilization. In some embodiments, the sheet of barrier material is produced from freezing the multi-layered dECM from porcine small intestine serosa product at −80° C. for a minimum of about 9 hours prior to lyophilization. In some embodiments, the sheet of barrier material is produced from freezing the multi-layered dECM from porcine small intestine serosa product at −80° C. for a minimum of about 10 hours prior to lyophilization. In some embodiments, the sheet of barrier material is produced from freezing the multi-layered dECM from porcine small intestine serosa product at −80° C. for a minimum of about 11 hours prior to lyophilization. In some embodiments, the sheet of barrier material is produced from freezing the multi-layered dECM from porcine small intestine serosa product at −80° C. for a minimum of about 12 hours prior to lyophilization. In some embodiments, the sheet of barrier material is produced from freezing the multi-layered dECM from porcine small intestine serosa product at −80° C. for a minimum of overnight prior to lyophilization.

In some embodiments, Serograft can be used in the peritoneal space in a subject that has undergone an abdominal surgery. In some embodiments, the Serograft can be applied in a subject with the material being placed in between the damaged tissue/organ surfaces. In some embodiments, the subject is a human. In some embodiments of the present disclosure, the biomaterial prevents post-operative peritoneal adhesions.

In another aspect, a powder comprising lyophilized solubilized dECM from porcine small intestine serosa is disclosed. In some embodiments, the powder can be used to prepare a hydrogel by resuspension with a liquid. In some embodiments, the solubilized dECM from porcine small intestine serosal is produced by treatment with pepsin. In such embodiments, the lyophilized solubilized dECM from porcine small intestine serosa has a sponge-like matrix. In some aspects, the powder is pulverized lyophilized pepsin-treated dECM from porcine small intestine serosa.

In yet another aspect, a hydrogel composition is disclosed. In some embodiments, the hydrogel composition comprises lyophilized solubilized dECM from porcine small intestine serosa and a liquid. The hydrogel composition is also referred to herein as “Serogel.” In some aspects, the osmolarity and ion concentrations of the liquid match that of the treatment subject's body, for example that of the human body. In other words, the liquid is isotonic to that of the treatment subject's body. In some embodiments, the liquid is a buffered saline solution. In particular embodiments, the liquid is a phosphate-buffered saline solution. In some embodiments, the Serogel may be used as a therapeutic delivery depot and/or as a scaffold for cell culture and tissue engineering. In some such applications, the hydrogel has neutral pH and is gel-like at temperature of at least 35° C.

In some aspects, a method of producing the dECM from porcine small intestine serosa is described. In some embodiments, the method comprises isolating a serosal layer from a porcine small intestine; incubating the isolated serosal layer from porcine small intestine in a solution comprising chloroform and methanol for at least 8 hours to produce a degreased serosal layer; incubating the degreased serosal layer with an enzyme solution for at least 8 hours to produce a partially decellularized serosal layer; incubating the partially decellularized serosal layer with a detergent solution under agitation for at least two hours to produce decellularized serosal layer; and incubating the decellularized serosal layer with peracetic acid solution followed by ethanol solution to produce dECM from porcine small intestine serosa. In some implementations, the serosal layer is mechanically dissociated from porcine small intestine. In some embodiments, the method further comprises a sterilization step. In some embodiments, the sterilization step comprises an ethylene oxide treatment.

In some aspects, the ratio of chloroform to methanol in the solution comprising chloroform and methanol treatment is 1:1 by volume. In particular implementations, the degreased serosal layer is produced from incubating the isolated serosal layer from porcine small intestine in the solution comprising chloroform and methanol overnight. In particular implementations, the degreased serosal layer is produced from incubating the isolated serosal layer from porcine small intestine in the solution comprising chloroform and methanol for about 10 hours. In particular implementations, the degreased serosal layer is produced from incubating the isolated serosal layer from porcine small intestine in the solution comprising chloroform and methanol for about 11 hours. In particular implementations, the degreased serosal layer is produced from incubating the isolated serosal layer from porcine small intestine in the solution comprising chloroform and methanol for about 12 hours. In particular implementations, the degreased serosal layer is produced from incubating the isolated serosal layer from porcine small intestine in the solution comprising chloroform and methanol for about 10-12 hours. In particular implementations, about 50 mL of the detergent solution is used for every 10-15 g wet tissue mass of the isolated serosal layer.

In some aspects, the enzyme solution comprises about 0.05%-about 0.5% trypsin. In some aspects, the enzyme solution comprises about 0.05% trypsin. In some aspects, the enzyme solution comprises about 0.1% trypsin. In some aspects, the enzyme solution comprises about 0.15% trypsin. In some aspects, the enzyme solution comprises about 0.2% trypsin. In some aspects, the enzyme solution comprises about 0.25% trypsin. In some aspects, the enzyme solution comprises about 0.3% trypsin. In some aspects, the enzyme solution comprises about 0.35% trypsin. In some aspects, the enzyme solution comprises about 0.4% trypsin. In some aspects, the enzyme solution comprises about 0.45% trypsin. In some aspects, the enzyme solution comprises about 0.5% trypsin. In some implementations, the enzyme solution is about 0.05%-about 0.5% trypsin in about 1—about 2.5 mM ethylenediaminetetraacetic acid (EDTA). In some implementations, the enzyme solution is about 0.05% trypsin in about 1—about 2.5 mM EDTA. In some implementations, the enzyme solution is about 0.1% trypsin in about 1—about 2.5 mM EDTA. In some implementations, the enzyme solution is about 0.15% trypsin in about 1—about 2.5 mM EDTA. In some implementations, the enzyme solution is about 0.2% trypsin in about 1—about 2.5 mM EDTA. In some implementations, the enzyme solution is about 0.25% trypsin in about 1—about 2.5 mM EDTA. In some implementations, the enzyme solution is about 0.3% trypsin in about 1—about 2.5 mM EDTA. In some implementations, the enzyme solution is about 0.35% trypsin in about 1—about 2.5 mM EDTA. In some implementations, the enzyme solution is about 0.4% trypsin in about 1—about 2.5 mM EDTA. In some implementations, the enzyme solution is about 0.45% trypsin in about 1—about 2.5 mM EDTA. In some implementations, the enzyme solution is about 0.5% trypsin in about 1—about 2.5 mM EDTA. In some implementations, the enzyme solution is 0.05% trypsin in about 2.21 mM EDTA. In some implementations, the enzyme solution is 0.1% trypsin in about 2.21 mM EDTA. In some implementations, the enzyme solution is 0.15% trypsin in about 2.21 mM EDTA. In some implementations, the enzyme solution is 0.2% trypsin in about 2.21 mM EDTA. In some implementations, the enzyme solution is 0.25% trypsin in about 2.21 mM EDTA. In some implementations, the enzyme solution is 0.3% trypsin in about 2.21 mM EDTA. In some implementations, the enzyme solution is 0.35% trypsin in about 2.21 mM EDTA. In some implementations, the enzyme solution is 0.4% trypsin in about 2.21 mM EDTA. In some implementations, the enzyme solution is 0.45% trypsin in about 2.21 mM EDTA. In some implementations, the enzyme solution is 0.5% trypsin in about 2.21 mM EDTA.

In some embodiments, the degreased serosal layer is incubated in the enzyme solution at about 37° C. overnight to produce the partially decellularized serosal layer. In some embodiments, the degreased serosal layer is incubated in the enzyme solution at about 37° C. for about 10 hours to produce the partially decellularized serosal layer. In some embodiments, the degreased serosal layer is incubated in the enzyme solution at about 37° C. for about 11 hours to produce the partially decellularized serosal layer. In some embodiments, the degreased serosal layer is incubated in the enzyme solution at about 37° C. for about 12 hours to produce the partially decellularized serosal layer. In some embodiments, the degreased serosal layer is incubated in the enzyme solution at about 37° C. for about 10-12 hours to produce the partially decellularized serosal layer. In particular implementations, about 50 mL of the enzyme solution is used for every about 10-about 15 g of wet tissue mass of the isolated serosal layer.

In some aspects, the detergent solution comprises about 0.1%—about 1% sodium dodecyl sulfate (SDS). In some aspects, the detergent solution comprises about 0.1% sodium SDS. In some aspects, the detergent solution comprises about 0.2% sodium SDS. In some aspects, the detergent solution comprises about 0.3% sodium SDS. In some aspects, the detergent solution comprises about 0.4% sodium SDS. In some aspects, the detergent solution comprises about 0.5% sodium SDS. In some aspects, the detergent solution comprises about 0.6% sodium SDS. In some aspects, the detergent solution comprises about 0.7% sodium SDS. In some aspects, the detergent solution comprises about 0.8% sodium SDS. In some aspects, the detergent solution comprises about 0.9% sodium SDS. In some aspects, the detergent solution comprises about 1% sodium SDS.

In some embodiments, the detergent solution comprises SDS in a NaCl solution. In some embodiments, the detergent solution comprises about 0.1%—about 1% SDS (w/v) in about 0.5%-1.5% NaCl (w/v) solution. In some embodiments, the detergent solution comprises about 0.1% SDS (w/v) in about 0.5%-1.5% NaCl (w/v) solution. In some embodiments, the detergent solution comprises about 0.2% SDS (w/v) in about 0.5%-1.5% NaCl (w/v) solution. In some embodiments, the detergent solution comprises about 0.3% SDS (w/v) in about 0.5%-1.5% NaCl (w/v) solution. In some embodiments, the detergent solution comprises about 0.4% SDS (w/v) in about 0.5%-1.5% NaCl (w/v) solution. In some embodiments, the detergent solution comprises about 0.5% SDS (w/v) in about 0.5%-1.5% NaCl (w/v) solution. In some embodiments, the detergent solution comprises about 0.6% SDS (w/v) in about 0.5%-1.5% NaCl (w/v) solution. In some embodiments, the detergent solution comprises about 0.7% SDS (w/v) in about 0.5%-1.5% NaCl (w/v) solution. In some embodiments, the detergent solution comprises about 0.8% SDS (w/v) in about 0.5%-1.5% NaCl (w/v) solution. In some embodiments, the detergent solution comprises about 0.9% SDS (w/v) in about 0.5%-1.5% NaCl (w/v) solution. In some embodiments, the detergent solution comprises about 1% SDS (w/v) in about 0.5%-1.5% NaCl (w/v) solution. In some embodiments, the detergent solution comprises about 0.1%-about 1% SDS (w/v) in about 0.5% NaCl (w/v) solution. In some embodiments, the detergent solution comprises about 0.1%-about 1% SDS (w/v) in about 0.6% NaCl (w/v) solution. In some embodiments, the detergent solution comprises about 0.1%—about 1% SDS (w/v) in about 0.7% NaCl (w/v) solution. In some embodiments, the detergent solution comprises about 0.1%—about 1% SDS (w/v) in about 0.8% NaCl (w/v) solution. In some embodiments, the detergent solution comprises about 0.1%—about 1% SDS (w/v) in about 0.9% NaCl (w/v) solution. In some embodiments, the detergent solution comprises about 0.1%—about 1% SDS (w/v) in about 1% NaCl (w/v) solution. In some embodiments, the detergent solution comprises about 0.1%—about 1% SDS (w/v) in about 1.1% NaCl (w/v) solution. In some embodiments, the detergent solution comprises about 0.1%—about 1% SDS (w/v) in about 1.2% NaCl (w/v) solution. In some embodiments, the detergent solution comprises about 0.1%—about 1% SDS (w/v) in about 1.3% NaCl (w/v) solution. In some embodiments, the detergent solution comprises about 0.1%—about 1% SDS (w/v) in about 1.4% NaCl (w/v) solution. In some embodiments, the detergent solution comprises about 0.1%—about 1% SDS (w/v) in about 1.5% NaCl (w/v) solution. In particular embodiments, the detergent solution is about 0.5% SDS (w/v) in about 0.9% NaCl (w/v) solution. In some implementations, the partially decellularized serosal layer is incubated in the detergent solution on an orbital shaker. In particular implementations, the partially decellularized serosal layer is incubated in the detergent solution on an orbital shaker rotating at about 50 rpm for about four hours to produce decellularized serosal layer. In particular implementations, about 50 mL of the detergent solution is used for every about 10—about 15 g of wet tissue mass of the isolated serosal layer.

In some embodiments, the dECM from the porcine small intestine serosa is produced from incubating the decellularized serosal layer with a peracetic acid solution for about 15—about 60 minutes followed by ethanol solution for about 15—about 60 minutes. In some embodiments, the dECM from the porcine small intestine serosa is produced from incubating the decellularized serosal layer with a peracetic acid solution for about 15 minutes followed by ethanol solution for about 15—about 60 minutes. In some embodiments, the dECM from the porcine small intestine serosa is produced from incubating the decellularized serosal layer with a peracetic acid solution for about 30 minutes followed by ethanol solution for about 15—about 60 minutes. In some embodiments, the dECM from the porcine small intestine serosa is produced from incubating the decellularized serosal layer with a peracetic acid solution for about 45 minutes followed by ethanol solution for about 15—about 60 minutes. In some embodiments, the dECM from the porcine small intestine serosa is produced from incubating the decellularized serosal layer with a peracetic acid solution for about 60 minutes followed by ethanol solution for about 15—about 60 minutes. In some embodiments, the dECM from the porcine small intestine serosa is produced from incubating the decellularized serosal layer with a peracetic acid solution for about 15—about 60 minutes followed by ethanol solution for about 15 minutes. In some embodiments, the dECM from the porcine small intestine serosa is produced from incubating the decellularized serosal layer with a peracetic acid solution for about 15—about 60 minutes followed by ethanol solution for about 30 minutes. In some embodiments, the dECM from the porcine small intestine serosa is produced from incubating the decellularized serosal layer with a peracetic acid solution for about 15—about 60 minutes followed by ethanol solution for about 45 minutes. In some embodiments, the dECM from the porcine small intestine serosa is produced from incubating the decellularized serosal layer with a peracetic acid solution for about 15—about 60 minutes followed by ethanol solution for about 60 minutes. In some embodiments, the dECM from the porcine small intestine serosa is produced from incubating the decellularized serosal layer with a peracetic acid solution for about 15 minutes followed by ethanol solution for about 30 minutes. In some embodiments, the dECM from the porcine small intestine serosa is produced from incubating the decellularized serosal layer with a peracetic acid solution for about 30 minutes followed by ethanol solution for about 30 minutes. In some embodiments, the dECM from the porcine small intestine serosa is produced from incubating the decellularized serosal layer with a peracetic acid solution for about 45 minutes followed by ethanol solution for about 30 minutes. In some embodiments, the dECM from the porcine small intestine serosa is produced from incubating the decellularized serosal layer with a peracetic acid solution for about 60 minutes followed by ethanol solution for about 30 minutes. In some embodiments, the dECM from the porcine small intestine serosa is produced from incubating the decellularized serosal layer with a peracetic acid solution for about 30 minutes followed by ethanol solution for about 15 minutes. In some embodiments, the dECM from the porcine small intestine serosa is produced from incubating the decellularized serosal layer with a peracetic acid solution for about 30 minutes followed by ethanol solution for about 45 minutes. In some embodiments, the dECM from the porcine small intestine serosa is produced from incubating the decellularized serosal layer with a peracetic acid solution for about 30 minutes followed by ethanol solution for about 60 minutes.

In some implementations, the peracetic acid solution comprises about 0.05%—about 0.25% peracetic acid (v/v). In some implementations, the peracetic acid solution comprises about 0.05% peracetic acid (v/v). In some implementations, the peracetic acid solution comprises about 0.1% peracetic acid (v/v). In some implementations, the peracetic acid solution comprises about 0.15% peracetic acid (v/v). In some implementations, the peracetic acid solution comprises about 0.2% peracetic acid (v/v). In some implementations, the peracetic acid solution comprises about 0.25% peracetic acid (v/v).

In some implementations, the ethanol solution comprises about 10%—about 40% ethanol (v/v). In some implementations, the ethanol solution comprises about 10% ethanol (v/v). In some implementations, the ethanol solution comprises about 20% ethanol (v/v). In some implementations, the ethanol solution comprises about 30% ethanol (v/v). In some implementations, the ethanol solution comprises about 40% ethanol (v/v).

In some implementations, about 25—about 100 mL of the peracetic acid solution or about 50 mL of the ethanol solution is used for every about 10—about 15 g of wet tissue mass of the isolated serosal layer. In particular implementations, about 25 mL of the peracetic acid solution or about 50 mL of the ethanol solution is used for every about 10—about 15 g of wet tissue mass of the isolated serosal layer. In particular implementations, about 50 mL of the peracetic acid solution or about 50 mL of the ethanol solution is used for every about 10—about 15 g of wet tissue mass of the isolated serosal layer. In particular implementations, about 75 mL of the peracetic acid solution or about 50 mL of the ethanol solution is used for every about 10—about 15 g of wet tissue mass of the isolated serosal layer. In particular implementations, about 100 mL of the peracetic acid solution or about 50 mL of the ethanol solution is used for every about 10—about 15 g of wet tissue mass of the isolated serosal layer.

In particular implementations, about 50 mL of the peracetic acid solution or about 25—about 100 mL of the ethanol solution is used for every about 10—about 15 g of wet tissue mass of the isolated serosal layer. In particular implementations, about 50 mL of the peracetic acid solution or about 25 mL of the ethanol solution is used for every about 10—about 15 g of wet tissue mass of the isolated serosal layer. In particular implementations, about 50 mL of the peracetic acid solution or about 75 mL of the ethanol solution is used for every about 10—about 15 g of wet tissue mass of the isolated serosal layer. In particular implementations, about 50 mL of the peracetic acid solution or about 100 mL of the ethanol solution is used for every about 10—about 15 g of wet tissue mass of the isolated serosal layer.

Throughout the entire method, the serosal layer remains substantially as a sheet of tissue. In some implementations, the method further comprises laying at least two sheets of dECM produce the Serograft from the from dECM from porcine small intestine serosa. In certain implementation of a method of producing the Serograft, the method further comprises chemically modifying the dECM from porcine small intestine serosa prior to laying the sheets of dECM.

Also described herein is a method of producing a hydrogel from porcine small intestine serosa. In some embodiments, the hydrogel is produced from dECM from porcine small intestine serosa after a pepsin digestion treatment. In some aspects, the pepsin digestion treatment utilizes a porcine pepsin. In some embodiments, the lyophilized Serograft can be subjected to digestion via a pepsin followed by lyophilization to form a dried spongy material. In some embodiments, the material can be pulverized, resuspended, and pH neutralized to form a gel-like substance.

In some aspects, the hydrogel is produced by pulverizing the tissue graft material to produce powdered decellularized serosal layer. In some embodiments, the powdered decellularized serosal layer is treated with an enzyme (for example, pepsin, papain, amylase, or collagenase) to produce a hydrogel precursor solution. The duration of enzyme treatment may be adjusted for achieving the desired physiochemical properties of the hydrogel. In some exemplary embodiments, increased digestion times with a pepsin lead to a more linear matrix architecture. In some exemplary embodiments, shorter digestion times with a pepsin lead to a more branched structure in the hydrogel. In some embodiments, having a more branched structure in the hydrogel leads to a mechanically robust hydrogel. In some embodiments, the hydrogel precursor solution is lyophilized to produce a sponge-like matrix. In some embodiments, the sponge-like matrix is pulverized to produce a hydrogel precursor powder. In some embodiments, a buffered saline solution is added to the hydrogel precursor powder to produce the hydrogel. In some embodiments, the buffered saline solution is PBS. In some aspects, a mixture containing the buffered saline solution and the hydrogel precursor powder is adjusted to a neutral pH (for example, about 7.4).

In certain implementations, the hydrogel is produced by isolating a serosal layer from a porcine small intestine and then subjecting isolated serosal layer to at least one treatment selected from the group consisting of: treatment with chloroform and methanol treatment, treatment with trypsin and EDTA, treatment with a detergent, and treatment with ethanol & peracetic acid thereby producing a decellularized serosal layer. In some embodiments, the method next comprises lyophilizing the decellularized serosal layer to produce a tissue graft material; pulverizing the tissue graft material to produce powdered decellularized serosal layer; and then treating the powdered decellularized serosal layer with pepsin to produce a hydrogel precursor solution. In some aspects, the pepsin treatment comprises combining a solution comprising 0.1-10 mg/mL porcine pepsin in 0.01M HCl with the powdered decellularized serosal layer. In some implementations, the pepsin solution comprises 0.1-5 mg/mL porcine pepsin, 0.1-2.5 mg/mL porcine pepsin, 0.1-2.5 mg/mL porcine pepsin, 0.5-1.5 mg/mL porcine pepsin, 0.5-1.0 mg/mL porcine pepsin, 0.75-1.5 mg/mL porcine pepsin, or about 1 mg/mL porcine pepsin. In some aspects, the pepsin solution comprises procine pepsin in 0.005-0.02M HCl, for example, 0.0075-0.015M HCl, 0.008-0.012M HCl, or about 0.01m HCl.

In some implementations, combination of the solution and powdered decellularized serosal layer is constantly stirred for 18-72 hours, for example, 24-60 hours, 36-50 hours, 40-50 hours, 45-50 hours, about 18 hours, about 24 hours, about 36 hours, about 48 hours, about 60 hours, or about 72 hours. The method of producing the hydrogel next comprises lyophilizing the hydrogel precursor solution to produce a sponge-like matrix; pulverizing the sponge-like matrix to produce a hydrogel precursor powder; and adding a buffered saline solution to the hydrogel precursor powder to produce the hydrogel. In some aspects, the combination of the buffered saline solution and the hydrogel precursor powder is adjusted to a neutral pH.

A method of reducing instance of post-operative intestinal adhesions in a subject is disclosed. The method comprises applying the barrier material, for example, the Serograft or Serogel, between the damaged tissue or organ surface and the peritoneum of the subject during abdominal surgery.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1. Method For Creating Decellularized Serosal Tissue and the Serograft Serosal decellularized ECM (dECM) biomaterial was generated by extracting the serosal layer from porcine small intestine tissue and subjecting it to a thorough decellularization procedure to ensure that any cell/cellular components are removed and only the decellularized ECM is left. The serosal layer from porcine small intestines was isolated via mechanical dissociation, decellularizing the tissue was achieved by subjecting it to various treatments like: chloroform and methanol treatment, trypsin and EDTA treatment, detergent treatment, ethanol and peracetic acid treatment for varying time periods, subjecting the resulting decellularized material to lyophilization to produce grafts, and sterilizing the generated grafts via ethylene oxide treatment for further usage.

Specifically, the decellularization protocol begins with cleaning the pig intestines by thoroughly rinsing with deionized water. The serosal layer was obtained from the intact intestines by manual removal (peeling) and collected in phosphate-buffered saline (PBS). Visible fat was removed from the serosa with the help of tweezers. Approximately 10 g of wet serosal layer was transferred into a 50 ml falcon tube and submerged in a chloroform: methanol solution (1:1 v/v) for overnight incubation (12 hours) inside a fume hood. Following incubation, the serosal material was rinsed thrice with deionized water thoroughly to remove any organic solvents. The serosal material was further incubated in 0.25% trypsin/2.21 mM EDTA solution at 37° C. overnight (12 hours). Next, the serosal material was rinsed thrice with PBS to remove the trypsin. The serosal material was further treated with a solution of 0.5% SDS (w/v) in 0.9% NaCl (w/v) solution for 4 hours on an orbital shaker at ˜50 RPM. The serosal material was then rinsed with PBS thrice to remove any residual detergent. Following this, the serosal material was soaked in 0.1% peracetic acid (v/v) for 30 mins then, 20% ethanol (v/v) for 30 mins. The serosal material was finally rinsed with PBS thrice to obtain the undigested serosal biomaterial.

To make the layered undigested serosal biomaterial, which is the Serograft, the decellularized tissue (still in the form of thin, translucent sheets, similar to the native tissue in physical form) was stretched onto a glass slide to form a uniformly and evenly spread layer. The glass slide can be of variable dimensions as per need. This step was repeated to create the desired number of layers. For example, the 8-layer Serogel requires repeating the step seven more times, with each layer of the decellularized tissue stretched to spread on top of the previous one. Finally, the multi-layered sheet was frozen at −80° C. for a minimum of 6 hours to overnight, followed by lyophilization to obtain the dried Serograft sheets.

Additional modifications can be used to cross-link sheets with every layer. For example, EDC/NHS chemistry can be used to conjugate carboxylic acids and amines to crosslink successive layers leading to stronger bonding between the multilayer structure.

### Example 2. Method From Creating Serogel

Serogel was prepared by pulverizing Serografts into a fine powder-like substance, treating the powdered Serograft with a solution containing 1 mg/ml porcine pepsin in 0.01M HCl under constant stirring at 60 RPM for 48 hours at room temperature, lyophilizing the obtained solution to form a sponge-like matrix, pulverizing the matrix, resuspending in 1× PBS and adjusting the pH to 7.4, and achieving gelation within 7-10 mins upon placing the above solution at 37° C.

In a specific protocol, the undigested serosal biomaterial obtained after the decellularization process was pulverized into a fine powder-like substance. The powdered dECM was then treated with a solution containing 1 mg/ml porcine pepsin in 0.01M HCl under constant stirring for 48 hours at room temperature. The resultant solution obtained after 48 hours was viscous and slightly milky in appearance. The solution was then lyophilized to form a sponge-like matrix. The matrix was further pulverized into a fine powder, followed by resuspension in 1× PBS and adjusting the pH to 7.4. The pre-gel solution (at various concentrations ranging from 2-15 mg/ml) upon incubation in 37° C. forms a hydrogel in about 10 minutes.

### Example 3. Swelling And Anti-Adhesive Behavior of Serografts

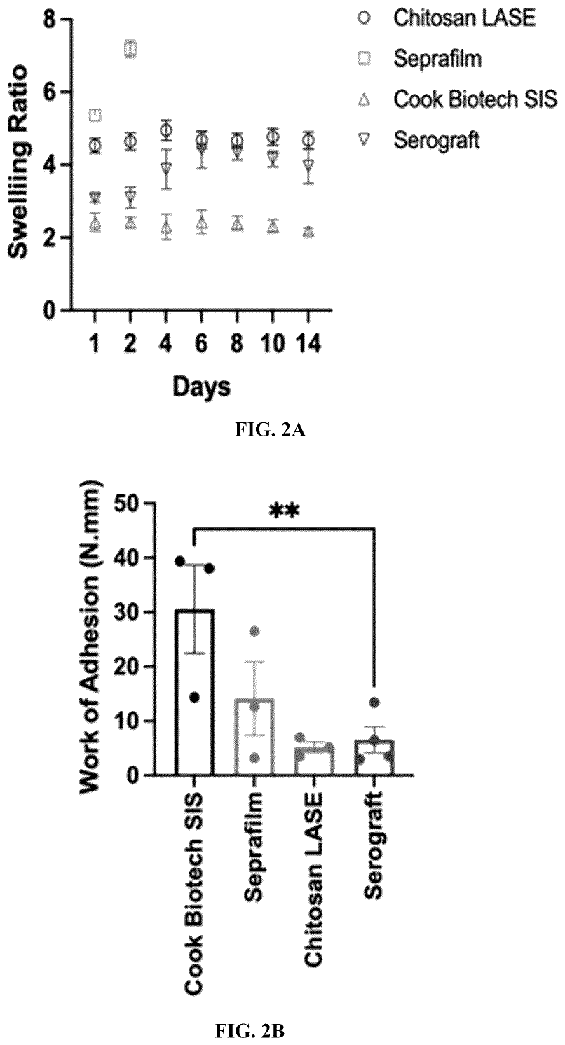

FIG. 2A discloses the swelling ratio of Serografts, a commercial dECM product from porcine small intestine submucosa (Cook Biotech™ SIS), a commercial anti-adhesive product (Seprafilm®), and a chistosan-based biomaterial film developed in the laboratory (Chitosan LASE) measured at predetermined timepoints. The materials were incubated in 1× PBS (pH about 7.4) at 37° C. in a humidified chamber on an orbital shaker under continuous stirring (about 50 RPM). FIG. 2B discloses the anti-adhesive behavior of Serografts measured with a texture analyzer ex vivo. Two sections of porcine small intestine were mounted on a template each and placed in between the clamps of the instrument opposing each other, with the respective test materials sandwiched between the intestine sections. Uniaxial tension was applied to one of the intestine sections and the force was recorded. Work of adhesion was defined as the total area under the curve of a force vs distance graph. Data represented are mean ±standard error of the mean of n≥3 independent experiments.

### Example 4. Ultimate Tensile Strength of and Young's Modulus of Serografts

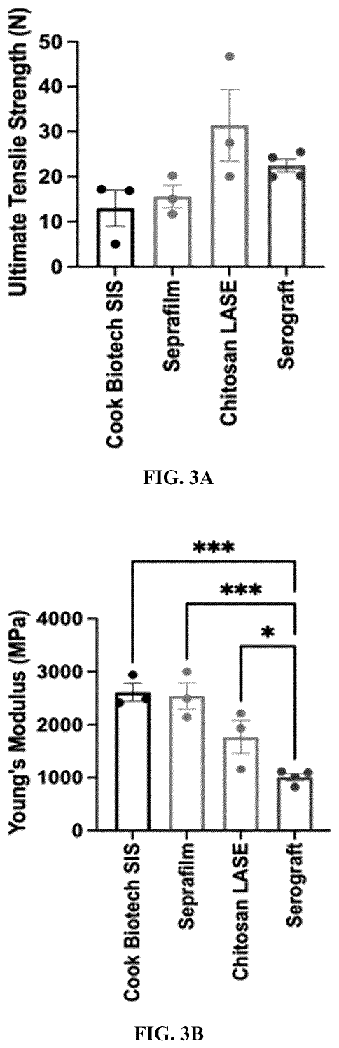

FIG. 3A shows Ultimate Tensile Strength (UTS) of Serografts, Cook Biotech™ SIS, Seprafilm®, and Chitosan LASE measured with a texture analyzer instrument. Differences in the means were not statistically significant. FIG. 3B shows Young's Modulus of Serografts, Cook Biotech SIS, Seprafilm, and Chitosan LASE measured with a texture analyzer instrument. Data represented are mean ±standard error of the mean of n≥3 independent experiments. Statistical significance was determined with One-way ANOVA followed by Fisher's LSD test.

### Example 5. Dynamic Mechanical Analysis of Serografts (DMA)

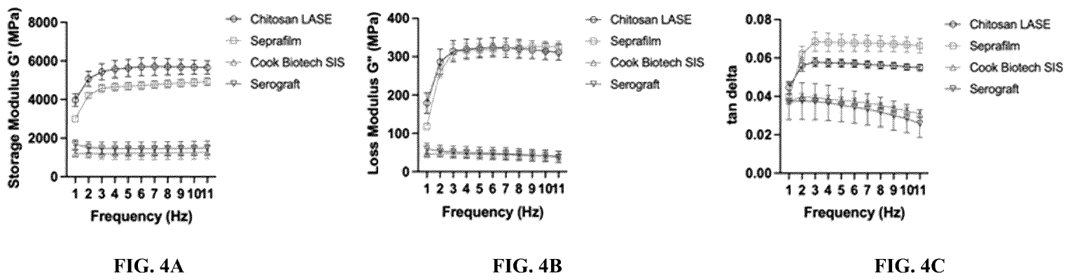

Storage Modulus of Serografts, Cook Biotech™ SIS, Seprafilm®, and Chitosan LASE was measured with a frequency sweep between 1-10 Hz under 0.1% strain, as shown in FIG. 4A. As illustrated in FIG. 4B, Loss Modulus of Serografts, Cook Biotech™ SIS, Seprafilm®, and Chitosan LASE was measured with a frequency sweep between 1-10 Hz under 0.1% strain. Additionally, tan δ (ratio of loss to storage modulus) of Serografts, Cook Biotech™ SIS, Seprafilm®, and Chitosan LASE was measured with a frequency sweep between 1-10 Hz under 0.1% strain (FIG. 4C). Data represented are mean ±standard error of the mean of n≥3 independent experiments.

### Example 6. Histological Analysis Of Serografts

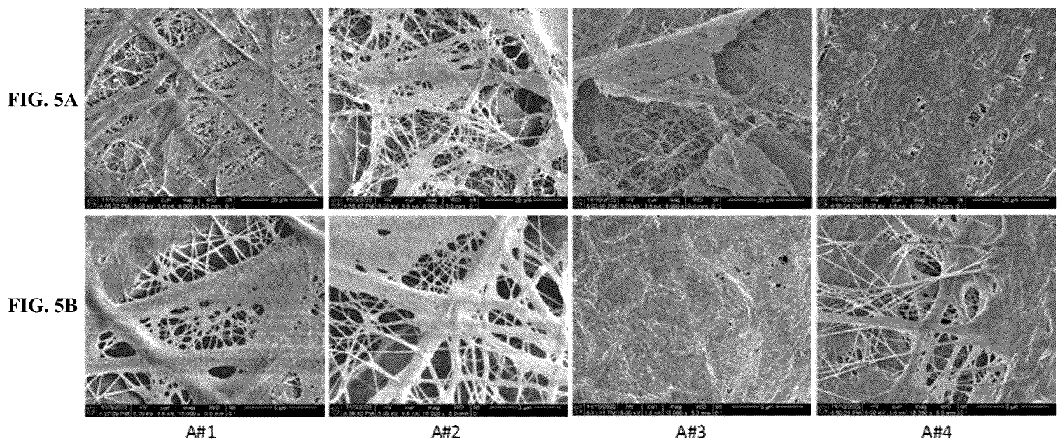

FIGS. 5A and 5B discloses non-limiting examples of scanning electron microscopy (SEM) of Serografts showing the presence of fibrillar structures of collagen at 4000× and 15000× magnification respectively.

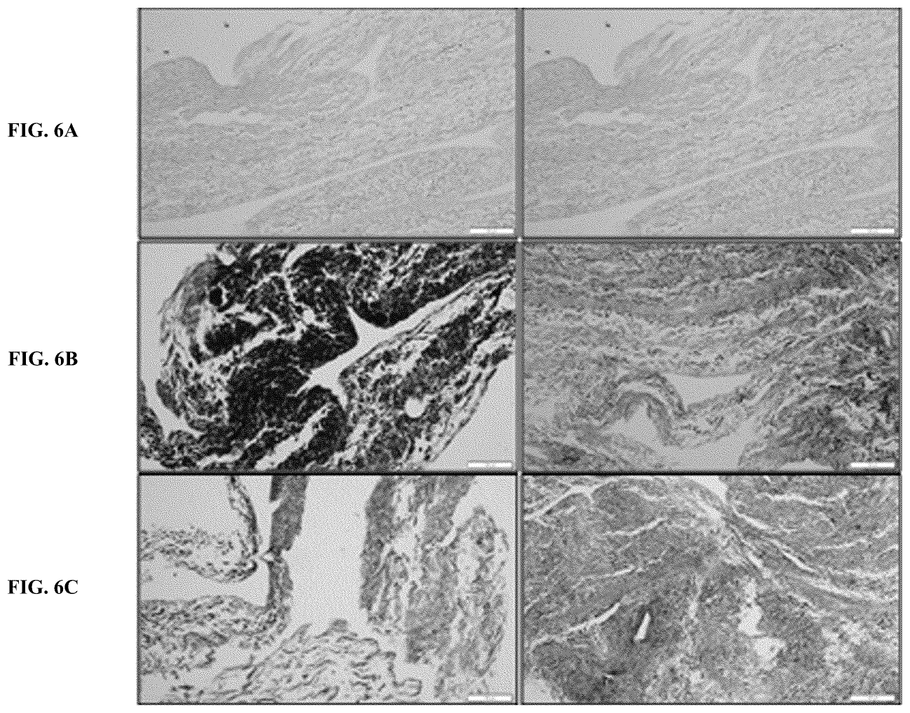

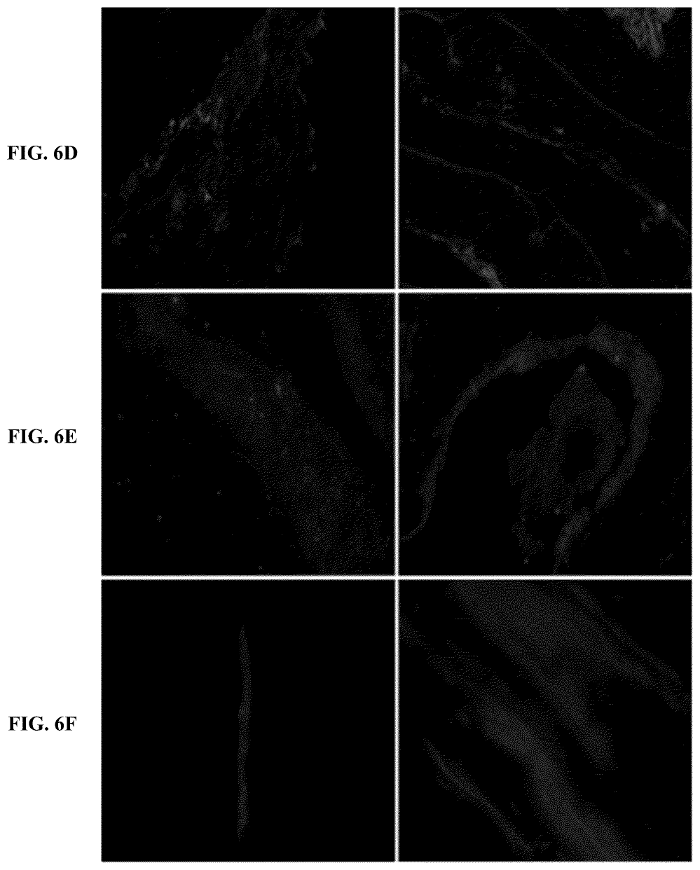

FIGS. 6A-6C illustrate examples of histological analysis with representative images of Serografts with Hematoxylin and Eosin (H&E) staining, with Masson's Trichrome staining indicating the presence of collagen, and with Alcian Blue staining indicating the presence of negatively charged glycosaminoglycans (GAGs) respectively. These images demonstrate successful decellularization. In another example, as shown in FIGS. 6D-6F, Collagen IV, Elastin and Laminin from the ECM were detected using Immunofluorescence (IF) indicating preservation of ECM proteins post-decellularization.

### Example 8. In Vivo Animal Model For Serograft Properties In Post-Operative Peritoneal Adhesions

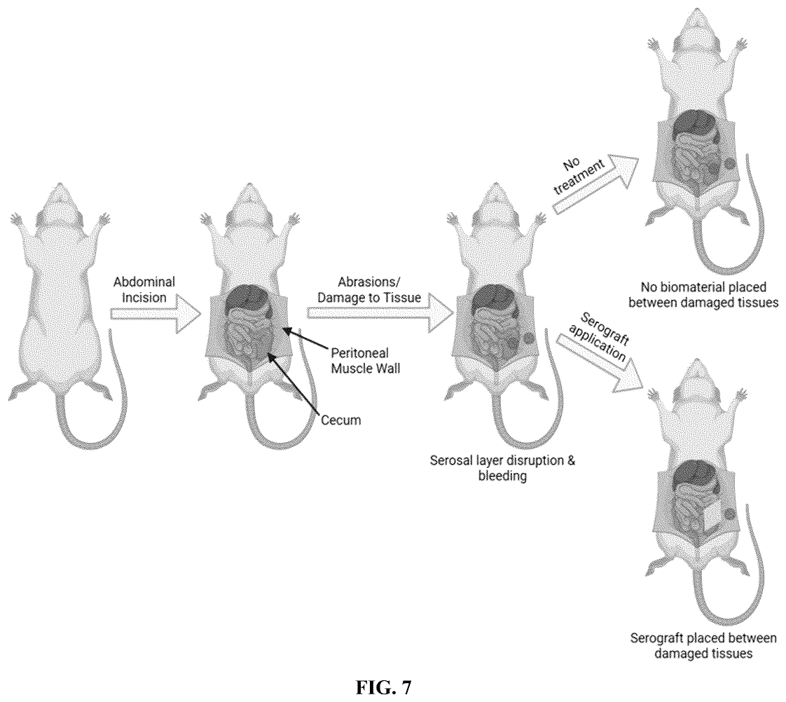

Sprague-Dawley rats at 8-10 weeks old were used for assessing the in vivo anti-adhesion potential of Serografts. For the surgical procedure, animals were anesthetized, placed on a heating pad in a supine position and the abdomen shaved using electric clippers. The shaved area was surgically prepped followed by creating a 2-3 cm long vertical midline incision in the abdomen. The cecum was gently exteriorized followed by abrasion of the cecal surface with the help of an abrasive pad. The adjacent peritoneal surface was abraded and a defect ˜2×2 cm was made in the abdominal muscle wall. A surgical grade polypropylene mesh was sutured into the defect. In the case of Serograft or Serogel usage, the materials will be placed between the abraded cecal surface and the polypropylene mesh. This will be followed by closing of the surgical site. FIG. 7 depicts the experimental plan.

### REFERENCES CITED

1. Golebiowska, et al., Decellularized extracellular matrix biomaterials for regenerative therapies: Advances, challenges and clinical prospects. Bioactive Materials, 2024, 32:98-123.
2. Luo, et al., A multi-step method for preparation of porcine small intestinal submucosa (SIS). Biomaterials, 2011, 32(3): 706-713.
3. Pouliot, et al., Porcine lung-derived extracellular matrix hydrogel properties are dependent on pepsin digestion time. Tissue Engineering Part C: Methods, 2020, 26(6): 332-346.
4. Almalla, et al., Papain-Based Solubilization of Decellularized Extracellular Matrix for the Preparation of Bioactive, Thermosensitive Pregels. Biomacromolecules, 2023, 24(12): 5620-5637.
5. Hoganson, et al., Preserved extracellular matrix components and retained biological activity in decellularized porcine mesothelium, Biomaterials, 2010, 31: 6934e6940

What is claimed:

1. A barrier material comprising at least two layers of decellularized extracellular matrix (dECM) from a porcine small intestine serosal tissue,
wherein the barrier material is a sheet comprising at least two layers of dECM from porcine small intestine serosa, and the layers of dECM are arranged on top of each other.

2. The barrier material of claim 1, wherein the sheet comprises 4-8 layers.

3. The barrier material of claim 1, wherein each layer of dECM from porcine small intestine serosa is conjugated with at least one of: a carboxylic acid, an amine, a hydrophilic polymer, and/or a combination thereof.

4. The barrier material of claim 1, wherein the sheet is lyophilized.

5. The barrier material of claim 3, wherein the sheet is lyophilized.

6. A method of decellularizing a serosal layer from a porcine small intestine, the method comprising:
isolating the serosal layer from the porcine small intestine;
incubating the isolated serosal layer in a solution comprising chloroform and methanol for at least 8 hours to produce a degreased serosal layer;
incubating the degreased serosal layer with an enzyme solution for at least 8 hours to produce a partially decellularized serosal layer;
incubating the partially decellularized serosal layer with a detergent solution under agitation for at least two hours to produce decellularized serosal layer; and
incubating the decellularized serosal layer with peracetic acid solution followed by ethanol solution to produce a decellularized extracellular matrix (dECM) from porcine small intestine serosa.

7. The method of claim 6, wherein the ratio of chloroform to methanol in the solution used to produce a degreased serosal layer is 1:1 by volume.

8. The method of claim 6, wherein the enzyme solution comprises 0.05-0.5% trypsin.

9. The method of claim 6, wherein the detergent solution comprises 0.1-1% sodium dodecyl sulfate (SDS).

10. The method of claim 6, wherein the partially decellularized serosal layer is incubated in the detergent solution on an orbital shaker.

11. A method of producing a barrier material, the method comprising
providing a decellularized extracellular matrix (dECM) from a porcine small intestine serosa produced according to the method of claim 6; and
layering the dECM from the porcine small intestine serosa to produce a multi-layered dECM product; and
lyophilizing the multi-layered dECM product to produce the barrier material.

12. The method of claim 11, further comprising:
producing a chemically modified dECM by conjugating the dECM from the porcine small intestine serosa with at least one of a carboxylic acid, an amine, a hydrophilic polymer, and/or a combination thereof,
wherein the chemically modified dECM is layered to produce the multi-layered dECM product.

13. The method of claim 11, further comprising subjecting the dECM from the porcine small intestine serosa to an ethylene oxide treatment.

14. The method of claim 11, wherein eight layers of the dECM from the porcine small intestine serosa are layered to produce the multi-layered dECM product.

15. A method of producing a hydrogel comprising:
providing a decellularized extracellular matrix (dECM) from a porcine small intestine serosa produced according to the method of claim 6;
pulverizing the dECM from the porcine small intestine serosa to produce a powdered decellularized serosal layer;
digesting the powdered decellularized serosal layer with an enzyme to produce a hydrogel precursor solution, wherein the enzyme is selected from pepsin, papain, amylase, or collagenase;
lyophilizing the hydrogel precursor solution to produce a sponge-like matrix; and
pulverizing the sponge-like matrix to produce a hydrogel precursor powder.

16. The method of claim 15, further comprising subjecting the dECM from the porcine small intestine serosa to an ethylene oxide treatment, thereby sterilizing the dECM from the porcine small intestine serosa.

17. The method of claim 15, further comprising adding a buffered saline solution to the hydrogel precursor powder to produce the hydrogel.

18. A hydrogel composition comprising:
a decellularized extracellular matrix derived from a porcine small intestine serosal tissue, wherein the decellularized extracellular matrix has been pulverized; and
a liquid,
wherein the hydrogel composition is gel-like at a temperature of at least 35° C.

19. The hydrogel composition of claim 18, wherein the buffered saline solution is a phosphate-buffered saline and the pH of the hydrogel composition is neutral.

20. A kit for preventing post-operative intestinal adhesions comprising decellularized extracellular matrix (dECM) from a porcine small intestine serosal tissue.
